(19) United States
(12) Patent Application Publication
(10) Pub. No.: US 2015/0366782 A1
(43) Pub. Date: Dec. 24, 2015

(54) LIPSTICK COMPOSITION HAVING IMPROVED GLOSS AND WEAR

(71) Applicant: L'OREAL, Paris (FR)

(72) Inventors: Hy Si BUI, Piscataway, NJ (US); Rita Jaky EL-KHOURI, Morristown, NJ (US)

(21) Appl. No.: 14/307,831

(22) Filed: Jun. 18, 2014

(51) Int. Cl. A61K 8/92; A61K 8/31; A61K 8/891; A61Q 1/06; A61K 8/81

(52) U.S. Cl. CPC A61K 8/92; A61Q 1/06; A61K 8/8141; A61K 8/891; A61K 8/31

(57) ABSTRACT

Disclosed is a lipstick composition comprising at least one silicone acrylate copolymer, at least one polypropylsilsesquioxane film forming resin, at least one polypropylsilsesquioxane wax substituted with alkyl units having at least 30 carbons, and at least one volatile hydrocarbon solvent, wherein ratio of the silicone acrylate copolymer to the polypropylsilsesquioxane resin is from about 1:1 to about 4:1.

### BACKGROUND OF THE INVENTION

The present invention relates to a lipstick composition comprising a polypropyl silsesquioxane film forming resin, a silicone acrylate copolymer, a volatile hydrocarbon solvent, a polypropylsilsesquioxane wax substituted with alkyl units having at least 30 carbons, a viscosity increasing agent, and a colorant, wherein the ratio of the silicone acrylate copolymer to the polypropylsilsesquioxane resin is from about 1:1 to about 4:1, said composition having a total amount of film forming polymers equal to or greater than 30%.

Currently, commercially available long wear lipsticks are typically comprised of a silicone resin, such as MQ resin, and a plasticizing agent. MQ resin-containing lipsticks are disclosed, for example, in U.S. Pat. No. 6,908,621. These products tend to have tacky feel.

The use of some silicone polymers in cosmetics, can improve comfort by providing an overall smooth feel. For example, polyorganosiloxane-containing polymers in cosmetic compositions, including lipsticks, are discussed in U.S. Pat. No. 7,879,316. The use of silicone acrylate copolymers and polypropylsilsesquioxane resins in liquid lipstick compositions has been described, for example, in US2007/0093619 and US2012/0301415. Typically, non-volatile solvents are used in these compositions to provide shine. However, the incorporation of these non-volatile solvents often reduces (compromises) wear properties.

Surprisingly, applicants have found that combining at least one polypropylsilsesquioxane film forming resin with at least one silicone acrylate copolymer in a particular ratio, together with a volatile hydrocarbon solvent and a polypropylsilsesquioxane wax substituted with alkyl units having at least 30 carbons, affords a long wear lipstick composition that is both more comfortable than MQ-based lip compositions and provides increased shine (gloss) without the need for additional non-volatile solvents.

### BRIEF SUMMARY OF THE INVENTION

The present invention relates to a cosmetic composition comprising:
(a) from about 15% to about 40% by weight of at least one silicone acrylate copolymer;
(b) from about 5% to about 25% by weight of at least one polypropylsilsesquioxane film forming resin;
(c) from about 0.1% to about 4% by weight of at least one polypropylsilsesquioxane wax substituted with alkyl units having at least 30 carbons;
(d) from about 15% to about 60% by weight of at least one volatile hydrocarbon solvent;
(e) optionally at least one colorant; and
(f) optionally at least one viscosity increasing agent;
wherein the weight percent ratio of the silicone acrylate copolymer (a) to the at least one polypropylsilsesquioxane film forming resin (b) is from about 1:1 to about 4:1; the total weight percent of the at least one silicone acrylate copolymer (a) plus the at least one polypropylsilsesquioxane film forming resin (b) is equal to or greater than about 30%; the weights being relative to the total weight of the composition.

The invention also relates to a method for making up the lips by applying to the lips the above composition.

### DETAILED DESCRIPTION OF THE INVENTION

It has been unexpectedly discovered that a cosmetic composition containing at least one silicone acrylate copolymer and at least one polypropylsilsesquioxane film forming resin in a ratio of from about 1:1 to about 4:1, together with a volatile solvent and a polypropylsilsesquioxane wax substituted with alkyl units having at least 30 carbons, provides a liquid lipstick composition with improved shine, improved wear, or both.

The lipstick compositions of the invention do not require, and preferably are free of non-volatile solvents.

“About” as used herein means within 10% of the indicated number (e.g. “about 10%” means 9%-11% and “about 2%” means 1.8%-2.2%).

“Additional non-volatile solvents” means non-volatile solvent other than that which may be associated with other components in the formula.

“At least one” means one or more and thus includes individual components as well as mixture/combinations.

“Comprising” it is meant that other steps and/or ingredients which do not affect the end result may be added. The products, compositions, methods and processes of the present invention can include all the essential elements and limitations of the invention described herein as well as any of the additional or optional ingredients, components, steps, or limitations described herein.

“Free” or “devoid” of as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of non-volatile solvents” means that non-volatile solvents are preferably omitted (that is 0% by weight), but can be present in the composition at an amount of less than about 0.25% by weight, typically less than about 0.1% by weight, typically less than about 0.05% by weight, based on the total weight of the composition as a whole.

The term “film forming” polymer means that the polymer is capable of forming a film, in particular a substantive film, on the lips, for example, after a solvent accompanying the film former has evaporated, absorbed into and/or dissipated on the lips.

By the term “film forming resin” it is meant that the polypropylsilsesquioxane film forming polymer is an amorphous polymer having a low Tg of from about 0° C. to about 5° C. and has substantive, film forming properties when applied to a keratinous material such as the lips.

The term “glass transition temperature” (Tg) generally refers to the temperature at which amorphous material changes from a glassy solid state to a rubbery state. The temperature may be measured by standard techniques in the art, such a Differential Scanning calorimetry (DSM), e.g., according to a standard protocol such as ASTM D3418-97 standard. The Tg of the film, such as those formed by incorporating (a) and (b) in the lipstick formulas of the invention, can be measured, for example by drawing down the composition on a glass plate using a draw dawn bar until the thickness of the resulting film is about 6 mil. The films are allowed to dry, peeled from the plate and then subjected to Differential Scanning calorimetry (DSC) scanning.

“Gloss” refers to the overall shine of a product once applied to a keratinous substrate, such as the lips. It is measured using a gloss meter and the units of measurement are gloss units (GU).

“INCI” is an abbreviation of International Nomenclature of Cosmetic Ingredients, which is a system of names provided by the International Nomenclature Committee of the Personal Care Products Council to describe personal care ingredients.

“Substituted” as used herein, means comprising at least one substituent. Non-limiting examples of substituents include atoms, such as oxygen atoms and nitrogen atoms, non-functional groups such as alkyl groups, as well as functional groups such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

The “wear” of compositions as used herein, refers to the extent by which the color of the composition remains the same or substantially the same as at the time of application, as viewed by the naked eye, after a certain period or an extended period of time. Wear properties may be evaluated by any method known in the art for evaluating such properties. For example, wear may be evaluated by a test involving the application of a composition to human hair, skin or lips and evaluating the color of the composition after a specified period of time. For example, the color of a composition may be evaluated immediately following application to skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours, and retains rich color even after eating.

All percentages, parts and ratios are based upon the total weight of the compositions of the present invention unless otherwise indicated.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

As used herein a range of ratios is meant to include every specific ratio within, and combination of subranges between, the given ranges.

According to various embodiments of the disclosure, the ratio of the weight percent of the at least one silicone acrylate copolymer (a) to the weight percent of the at least one polypropylsilsesquioxane film forming resin (b) is from about 1:1 to about 4:1, such as from about 1.5:1 to about 3:1, and from about 2:1 to about 4:1, and from about 3:1 to about 4:1.

In a particular embodiment the ratio of (a) to (b) is from about 2:1 to about 4:1.

In various embodiments, the weight percent ratio of (a) to (b) is about 1:1, or about 1.5:1, or about 2:1 or about 2.3:1, or about 3:1, or about 4:1.

In a particular embodiment, the weight percent ratio of (a) to (b) is about 2.3:1.

In an embodiment the present invention relates to a cosmetic composition comprising:
(a) from about 15% to about 40% by weight of at least one silicone acrylate copolymer;
(b) from about 5% to about 25% by weight of at least one polypropylsilsesquioxane film forming resin;
(c) from about 0.1% to about 4.0% by weight of at least one polypropylsilsesquioxane wax substituted with alkyl units having at least 30 carbons;
(d) from about 15% to about 60% by weight of at least one volatile hydrocarbon solvent;
(e) optionally least one colorant; and
(f) optionally at least one viscosity increasing agent;
wherein the weight percent ratio of the silicone acrylate copolymer (a) to the at least one polypropylsilsesquioxane film forming resin (b) is from about 1:1 to about 4:1; and the total weight percent of the at least one silicone acrylate copolymer (a) plus the at least one polypropylsilsesquioxane film forming resin (b) is equal to or greater than about 30%; the weights being relative to the total weight of the composition.

In another embodiment the invention relates to a composition comprising:
(a) from about 15% to about 40% by weight of at least one silicone acrylate copolymer;
(b) from about 5% to about 25% by weight of at least one polypropylsilsesquioxane film forming resin;
(c) from 0.1% to about 4% by weight of at least one polypropylsilsesquioxane wax substituted with alkyl units having at least 30 carbons;
(d) from about 15% to about 60% by weight of at least one volatile hydrocarbon solvent;
(e) from about 0.5% to about 18% of least one colorant; and
(f) from about 1% to about 20% of at least one viscosity increasing agent;
wherein the weight percent ratio of the silicone acrylate copolymer (a) to the at least one polypropylsilsesquioxane film forming resin (b) is from about 1:1 to about 4:1; the total weight percent of the at least one silicone acrylate copolymer (a) plus the at least one polypropylsilsesquioxane film forming resin (b) is equal to or greater than about 30%; the weights being relative to the total weight of the composition.

In another embodiment the invention relates to a method of improving the shine of a lipstick composition comprising including in said composition:
(a) from about 15% to about 40% by weight of at least one silicone acrylate copolymer;
(b) from about 5% to about 25% by weight of at least one polypropylsilsesquioxane film forming resin;
(c) from 0.1% to about 4% by weight of at least one polypropylsilsesquioxane wax substituted with alkyl units having at least 30 carbons;
(d) from about 15% to about 60% by weight of at least one volatile hydrocarbon solvent;
(e) from about 0.5% to about 18% of least one colorant; and
(f) optionally from about 1% to about 20% of at least one viscosity increasing agent;
wherein the weight percent ratio of the silicone acrylate copolymer (a) to the at least one polypropylsilsesquioxane film forming resin (b) is from about 2:1 to about 4:1; the total weight percent of the at least one silicone acrylate copolymer (a) plus the at least one polypropylsilsesquioxane film forming resin (b) is equal to or greater than about 30%; the weights being relative to the total weight of the composition.

In another embodiment the weight percent ratio of the silicone acrylate copolymer (a) to the at least one polypropylsilsesquioxane film forming resin (b) is from about 1.5:1 to about 4:1.

In another embodiment the invention relates to a method of improving the wear of a lipstick composition comprising including in said composition:
(a) from about 15% to about 40% by weight of at least one silicone acrylate copolymer;
(b) from about 5% to about 25% by weight of at least one polypropylsilsesquioxane film forming resin;
(c) from 0.1% to about 4% by weight of at least one polypropylsilsesquioxane wax;
(d) from about 15% to about 60% by weight of at least one volatile hydrocarbon solvent;
(e) from about 0.5% to about 18% of least one colorant; and
(f) from about 1% to about 20% of at least one viscosity increasing agent;
wherein the weight percent ratio of the silicone acrylate copolymer (a) to the at least one polypropylsilsesquioxane film forming resin (b) is from about 1:1 to about 3:1; the total weight percent of the at least one silicone acrylate copolymer (a) plus the at least one polypropylsilsesquioxane film forming resin (b) is equal to or greater than about 30%; the weights being relative to the total weight of the composition.

In another embodiment the sum of the weight percent of the at least one silicone acrylate copolymer (a) plus the at least one polypropylsilsesquioxane film forming resin (b) is from about 30% to about 60%, or from about 35% to about 55%, or from about 40% to about 50%. Having a total amount of film forming resins at equal to or greater than 30% improves the wear of the composition.

### The Silicone Acrylate Copolymer (a)

The compositions of the present invention comprise at least one silicone acrylate copolymer.

The at least one silicone acrylate copolymer polymer can be chosen from silicone/(meth)acrylate copolymers, such as those as described in U.S. Pat. Nos. 5,061,481, 5,219,560, 5,262,087 and US 2012/0301415, the entire contents of which are hereby incorporated by reference. They may also be selected from polymers derived from non-polar silicone copolymers comprising repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the products sold under the tradenames KP-545 (cyclopentasiloxane (and) acrylates/dimethicone copolymer), KP-543 (butyl acetate (and) acrylates/dimethicone copolymer), KP-549 (methyl trimethicone (and) acrylates/dimethicone copolymer), KP-550 (tentative INCI name: isododecane (and) acrylate/dimethicone copolymer), and mixtures thereof. Additional examples include the acrylate/dimethicone copolymers sold by Dow Corning under the tradenames FA 4001 CM SILICONE ACRYLATE (cyclopentasiloxane (and) acrylates/polytrimethylsiloxymethacrylate copolymer) and FA 4002 ID SILICONE ACRYLATE (isododecane (and) acrylates/polytrimethylsiloxymethacrylate Copolymer), and mixtures thereof.

Further non-limiting examples include polymers comprising a backbone chosen from vinyl polymers, methacrylic polymers, and acrylic polymers and at least one chain chosen from pendant siloxane groups and pendant fluorochemical groups. Non-limiting examples of such polymers and their synthesis are disclosed, for example, in U.S. Pat. Nos. 4,972,037, 5,061,481, 5,209,924, 5,849,275, and 6,033,650, and WO 93/23446, WO 95/06078 and WO 01/32737, the disclosures of which are hereby incorporated by reference. These polymers may be sourced from various companies. One such company is Minnesota Mining and Manufacturing Company which offers these types of polymers under the tradenames “Silicone Plus” polymers (for example, poly(isobutyl methacrylate-co-methyl FOSEA)-g-poly(dimethylsiloxane), sold under the tradename SA 70-5 IBMMF).

Other non-limiting examples of useful silicone acrylate polymers include silicone/acrylate graft terpolymers, for example, the copolymers described in WO 01/32727 A1, the disclosure of which is hereby incorporated by reference.

According to other embodiments, the polymer comprises a backbone chosen from vinyl backbones, methacrylic backbones, and acrylic polymeric backbones and further comprises at least one pendant siloxane group. Non-limiting examples of such polymers are disclosed in U.S. Pat. Nos. 4,693,935, 4,981,903, and 4,981,902, the disclosures of which are hereby incorporated by reference.

Other useful polymers include those described in U.S. Pat. No. 5,468,477, the disclosure of which is hereby incorporated by reference. A non-limiting example of these polymers is poly(dimethylsiloxane)-g-poly(isobutyl methacrylate), which is commercially available from 3M Company under the tradename VS 70 IBM.

In an embodiment the silicone acrylate copolymer has a glass transition temperature (Tg) above 20° C.

In an embodiment, the silicone acrylate copolymer is an acrylates/dimethicone copolymer having a Tg above 20° C.

The silicone acrylate polymers may be present in the composition of the invention in an amount ranging from about 15% to about 40% by weight, particularly from about 18% to about 33% by weight, more preferably from about 19% to about 30% by weight, and more preferably from about 20% to about 29% by weight, including all ranges and subranges therebetween, relative to the total weight of the composition.

### The Polypropylsilsesquioxane Film Forming Resin (b)

The compositions of the present invention comprise at least one polypropyl silsesquioxane film forming resin.

Silsesquioxane resins are a specific form of silicone resin. Silicone resin nomenclature is known in the art as “MDTQ” nomenclature, whereby a silicone resin is described according to the various monomeric siloxane units which make up the polymer. Each letter of “MDTQ” denotes a different type of unit. When the film forming resin is made up predominantly of tri-functional units (or T units), it is generally called a silsesquioxane resin. See, US2006/0292096.

Examples of silsesquioxane resins that may be used in the present invention are alkyl silsesquioxane resins that are silsesquioxane homopolymers and/or copolymers having an average siloxane unit of the general formula R1n SiO(4−n)/2, wherein each R1 is a propyl group, wherein more than 80 mole % of R1 represent a C3-C10 alkyl group, n is a value of from 1.0 to 1.4, and more than 60 mole % of the copolymer comprises R1SiO3/2 units. As each R1 is a propyl group these polymers are called polypropylsilsesquioxane resins or “t-propyl” silsesquioxane resins. These resins and methods of making them are described, for example in U.S. Pat. No. 8,586,013, 2012/0301415, 2007/0093619, and 2006/0292096, all of which are herein incorporated by reference.

A non-limiting example of a polypropylsilsesquioxane resin suitable for use in the present invention is commercially available from Dow Corning as Dow Corning 670 Fluid and Dow Corning 680 Fluid. Dow Corning 670 and 680 Fluids have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises RSiO_{3/2} units, and having a hydroxyl or alkoxy content from 0.2 to 10% by weight, for example between 1 and 4% by weight, preferably between 5 and 10% by weight, and more preferably between 6 and 8% by weight. Preferably, the polypropylsilsesquioxane resin has a molecular weight from about 5000 to about 30,000 and a Tg from about −5° C. to about 5° C.

The film forming polypropylsilsesquioxane resin (b) may be present in the instant compositions in an amount ranging from about 5% to about 40% by weight, preferably from about 7% to about 25% by weight, more preferably from about 10% to about 22% by weight, including all ranges and subranges therebetween, relative to the total weight of the composition.

### Polypropylsilsesquioxane Wax (c)

The cosmetic compositions of the present invention also contain at least one polypropylsilsesquioxane wax.

Certain polypropylsilsesquioxane waxes are discussed in U.S. Pat. No. 7,482,419, the entire content of which is hereby incorporated by reference. These compounds are also sometimes referred to as polypropylsilsesquioxane resin waxes.

Not all polypropylsilsesquioxane waxes yield stable colored cosmetic products. More particularly, it has been found that only those polypropylsilsesquioxane waxes substituted with alkyl units having at least 30 carbons are stable.

The polypropylsilsesquioxane wax useful in the compositions of the invention comprises at least 40 mole % of siloxy units having the formula (R2R′SiO1/2)×(C3H7SiO3/2)y, where x and y have a value of 0.05 to 0.95, R2 is an alkyl group having from 1 to 8 carbon atoms, and R′ is a monovalent hydrocarbon having 30 to 40 carbon atoms and greater. As used herein, x and y represent the mole fraction of (R2R′SiO1/2) and (C3H7SiO3/2) siloxy units relative to each other present in the polypropylsilsesquioxane wax. Thus, the mole fraction of (R2R′SiO1/2) and (C3H7SiO3/2) siloxy units each can independently vary from 0.05 to 0.95. Preferably R is a methyl, and R′ is an alkyl having at least 30 carbons.

Typically, the value of x is 0.05 to 0.95, or alternatively, 0.2 to 0.8, the value of y is 0.05 to 0.95, alternatively 0.2 to 0.8. However, the combination of (R2R′SiO1/2) and (C3H7SiO3/2) siloxy units present should preferably total at least 40 mole %, alternatively 60 mole %, or alternatively 90 mole % of all siloxy units present in the polypropylsilsesquioxane wax.

The number average molecular weight of the polypropylsilsesquioxane wax substituted with alkyl units having at least 30 carbons typically ranges from about 750 to about 10,000, such as from about 1,000 to about 5,000.

Preferably, the polypropylsilsesquioxane wax substituted with alkyl units having at least 30 carbons has a melting temperature (Tm) from about 60° C. to about 80° C.

A particularly preferred polypropylsilsesquioxane wax for use in the present invention is a C30-45 alkyldimethylsilyl polypropylsilsesquioxane commercially available from DOW CORNING under the tradename SW-8005 C30 Resin Wax (C30-45 alkyldimethylsilyl polypropylsilsesquioxane (and) paraffin wax).

The polypropylsilsesquioxane wax substituted with alkyl units having at least 30 carbons is generally present in the cosmetic composition of the present invention in an amount ranging from about 0.1% to about 4% by weight; such as from about 0.3% to about 3% by weight; such as from about 0.4% to about 2% by weight, all weights being based on the weight of the composition as a whole. In a particular embodiment, the polypropylsilsesquioxane wax substituted with alkyl units having at least 30 carbons is generally present in the cosmetic composition of the present invention in an amount ranging from about 0.6%.

In an embodiment the compositions of the invention include a low amount of wax (that is not greater than about 2%).

### The Volatile Hydrocarbon Solvent (d)

The cosmetic compositions of the present invention also contain at least one volatile hydrocarbon solvent

### Volatile Solvents

As used herein “volatile solvent” means any non-aqueous medium capable of evaporating on contact with the skin or the lips in less than one hour at room temperature and atmospheric pressure. “Volatile” solvents typically have a flash point of less than about 100° C.

Non-limiting examples of suitable volatile hydrocarbon solvents include volatile hydrocarbon-based oils having from 8 to 16 carbon atoms, and mixtures thereof, and in particular branched C8 to C16 alkanes such as C8 to C16 isoalkanes (also known as isoparaffins), such as isododecane, isodecane, isohexadecane, which are commercially available under the trade names of Isopar or Permethyl. Also useful are C8 to C16 branched esters, such as isohexyl or isodecyl neopentanoate, as well as alcohols, and mixtures of these compounds. Preferably, the volatile hydrocarbon-based oils have a flash point of at least 40° C.

Examples of volatile hydrocarbon-based oils include, but are not limited to those given in Table 1 below.

TABLE 1

| | Flash Point |
|---|---|
| Compound | (° C.) |
| Isododecane | 43 |
| Isohexadecane | 102 |
| Propylene glycol n-butyl ether | 60 |
| Ethyl 3-ethoxypropionate | 58 |
| Propylene glycol methylether acetate | 46 |
| Isopar L (isoparaffin C11-C13) | 62 |
| Isopar H (isoparaffin C11-C12) | 56 |

In an embodiment, the solvent is a volatile solvent selected from isododecane, isooctane, isodecane, isohexadecane, and mixtures thereof.

The volatile solvent may also be chosen from volatile silicone oils, which may be linear or cyclic, having a viscosity at room temperature typically less than or equal to 6 centiSotkes (cSt), and having from 2 to 7 silicon atoms, optionally substituted with alkyl or alkoxy groups of 1 to 10 carbon atoms.

Examples of suitable volatile silicone oils include, but are not limited to, those listed in Table 2 below.

TABLE 2

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

The at least one volatile hydrocarbon solvent is generally present in the cosmetic composition of the present invention in an amount ranging from about 15% to about 60% by weight, typically from about 5% to about 70% by weight, including from about 25% to about 55% by weight, more typically from about 40% to about 50% by weight; including all ranges and subranges therebetween, all weights being based on the weight of the composition as a whole.

### Non-Volatile Solvent (Optional)

While the compositions of the invention may be free of non-volatile solvents, in an embodiment the cosmetic compositions optionally may include a volatile hydrocarbon solvent.

Non-limiting examples of suitable non-volatile solvents include, but are not limited to, various types of oils. These oils include hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, corn oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl alcohol or linolenyl alcohol, isostearyl alcohol or octyldodecanol; silicone oils such as polydimethylsiloxanes (PDMS), which are optionally phenylated such as phenyltrimethicones, trimethyl pentaphenyl siloxane, tetramethyl tetraphenyl siloxane, or optionally substituted with aliphatic and/or aromatic groups that are optionally fluorinated, or with functional groups such as hydroxyl, thiol and/or amine groups; polysiloxanes modified with fatty acids, with fatty alcohols, with polyoxyalkylenes or with hydrocarbyl functional groups, fluorosilicones and perfluoro oils.

When present, the non-volatile solvent is generally present in the cosmetic composition of the present invention in an amount ranging from about 5% to about 30% by weight, most typically from about 10% to about 20%, including all ranges and subranges therebetween, all weights being based on the weight of the composition as a whole.

### Colorant (e) (Optional)

The cosmetic compositions of the present invention optionally may contain at least one cosmetically acceptable colorant such as a pigment or dyestuff.

Non-limiting examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface-treated within the scope of the present invention but are not limited to treatments such as silicones, perfluorinated compounds, lecithin, and amino acids.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570), and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride, and mixtures thereof.

When a colorant is present, the precise amount and type of colorant employed in the compositions of the present invention will depend on the color, intensity and use of the cosmetic composition and, as a result, will be determined by those skilled in the art of cosmetic formulation. However, in an embodiment the compositions include a colorant in an amount of from about 0.5% to about 18%, by weight, more typically from about 3% to about 15% by weight, including all ranges and subranges therebetween, relative to the total weight of the composition.

### The Viscosity Increasing Agent (f) (Optional)

The cosmetic compositions of the present invention optionally may also contain one or more viscosity increasing agents.

Representative viscosity increasing agents include thickening agents, rheology modifying agents and gelling agents. The viscosity increasing agent(s) that may be useful in the practice of embodiments of the disclosure include those conventionally used in cosmetics such as polymers of natural origin and synthetic polymers.

Viscosity increasing agents may be selected from, for example vegetable gums, liposoluble/lipodispersible polymers, salts, and mixtures thereof. Preferably the viscosity increasing agents are not waxes.

Representative viscosity increasing agents that may be used in the practice of embodiments according to the disclosure may be chosen from nonionic, anionic, cationic, and amphoteric polymers, including acrylate-based polymers, polysaccharides, polyamino compounds, amphiphilic polymers, and other viscosity modifiers such as cellulose-based thickeners (e.g., hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, cross-linked homopolymers of acrylic acid or of acrylamidopropane-sulfonic acid, associative polymers, non-associative thickening polymers, water-soluble thickening polymers, and mixtures of these.

Other non-limiting examples of such agents include, glycerol behenate, polyethylene and copolymers thereof such as PEG-150 distearate, magnesium stearate, synthetic polymers such as polyacrylic acid (available commercially as Carbomers) and acrylates copolymers such as sodium polyacrylate and polyacryloyldimehtyl taurate, and mixtures of these.

### Gelling Agents

In an embodiment, the composition of the invention includes at least one viscosity increasing agent selected from a gelling agent. Gelling agents useful in cosmetic compositions are described, for example, in US 2012/0301415 and U.S. Pat. No. 8,637,057, both which are herein incorporated by reference.

The at least one gelling agent may be chosen from gelling agents in polymeric form and gelling agents in mineral form.

In an embodiment the gelling agent is selected from modified clays. Non-limiting examples of useful modified clays include hectorites modified with an ammonium chloride of a C₁₀ to C₂₂ fatty acid. Examples of such gellants are hectorite modified with distearyldimethylammonium chloride, also known as quaternium-18 bentonite, such as the products marketed under the brand name Bentone 34 (by the company Rheox), or the products Claytone XL, Claytone 34 and Claytone 40 (by the company Southern Clay), the modified clays commonly referred to as quaternium-18 benzalkonium bentonites sold or made under the names Claytone HT, Claytone GR and Claytone PS (by the company Southern Clay), the clays modified with stearyldimethylbenzoylammonium chloride, known as steralkonium bentonites, such as the products sold or made under the brand names Claytone APA and Claytone AF (by the company Southern Clay), or Baragel 24 (available from Rheox).

In a particular embodiment the gelling agent is disteardimonium hectorite, such as that sold by Elementis under the name Bentone Gel (disteardimonium hectorite (and) propylene carbonate).

Other useful mineral gelling agents include silica, such as fumed silica. The fumed silica may have a particle size, which may be nanometric to micrometric, for example ranging from about 5 nm to 200 nm.

The fumed silicas may be obtained by high-temperature hydrolysis of a volatile silicon compound in a hydrogen-oxygen flame, producing a finely divided silica. This process makes it possible to obtain hydrophilic silicas that have a large number of silanol groups at their surface. Non-limiting examples of hydrophilic silicas, all of which have the INCI name “silica” include the following products having the following tradenames: “Aerosil 130®”, “Aerosil 200®” “Aerosil 255®”, “Aerosil 300®” and “Aerosil 380®,” available the company Degussa; and “CAB-O-SIL HS-5®”, “CAB-O-SIL EH-5®”, “CAB-O-SIL LM-130®”, “CAB-O-SIL MS-55®” and “CAB-O-SIL M-5®,” available from the company Cabot.

In the event that a viscosity increasing agent is employed, for example such as a gelling agent, it may be present in the composition in an amount of from about 1% to about 30% by weight, preferably from about 2% to 20% by weight, and more preferably from 5% to 15% by weight, based on the weight of the composition.

### Additional Optional Additives/Auxiliary Agents

The compositions of the present invention may further comprise any cosmetically or dermatologically acceptable additional additives such as additional thickeners/viscosity increasing agents, additional film formers, plasticizers, antioxidants, essential oils, preserving agents, fragrances, fillers, pasty fatty substances, additional waxes, neutralizing agents, emollients, moisturizers, vitamins, essential fatty acids, sunscreens, surfactants, medicaments, and mixtures thereof. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These additives may be present in the composition in a proportion from 0% to 99% (such as from 0.01% to 90%) relative to the total weight of the composition and further such as from 0.1% to 50% (if present), including all ranges and subranges therebetween.

It has been surprisingly discovered that the association of at least one polypropylsilsesquioxane film forming resin with at least one silicone acrylate copolymer in a particular ratio, together with a volatile solvent and a polypropylsilsesquioxane wax substituted with alkyl units having at least 30 carbons, affords a long wear lipstick composition that is both more comfortable and/or provides increased gloss. In an embodiment this is achieved even without additional non-volatile solvents.

The compositions of the present invention are useful as compositions for making up the skin, in particular the lips.

The present invention will be better understood from the examples which follow, all of which are intended for illustrative purposes only.

### Examples

Lipstick compositions in accordance with the present invention as well as comparative and control compositions were prepared as described below. The ingredients employed in each example are provided in Table 3.

TABLE 3
Sample Lip Compositions (Liquid)

| | | Example 1 | Example 2 | | | |
|---|---|---|---|---|---|---|
| | Comparative 1 | (Amt %) | (Amt %) | | | |
| Compound | (Amt %) | (improved wear) | (improved shine) | Control 1 | Control 2 | Comparative 2 |
| MQ/PSPA¹ | 0 | 0 | 0 | 0 | 0 | 40 |
| | | | | | | (24/18) |
| acrylates/dimethicone | 12 | 20 | 28 | 0 | 40 | 0 |
| copolymer (40% active) (a) | 18 | 30 | 38.67 | | 43.33 | |
| (60% solvent-isododecane) | | | | | | |
| (KP550, Shin Etsu) | | | | | | |
| poly propylsislesquioxane | 27.99 | 19.96 | 12 | 39.6 | 0 | 0 |
| (72%) (b) | 10.89 | 7.76 | 4.67 | 15.4 | | |
| (and) isododecane (28%) | | | | | | |
| (Dow Corning 680 ID Fluid) | | | | | | |
| C30-45 alkyldimethylsilyl | 0.6 | 0.6 | 0.6 | 0.6 | 0.6 | 0.6 |
| polypropylsilsesquioxane (and) | | | | | | |
| paraffin wax (99.7%/0.3%) (c) | | | | | | |
| (Dow Corning SW-8005 C30 | | | | | | |
| Resin Wax) | | | | | | |
| isododecane (d) QS | 10.31 | 10.47 | 4.86 | 36.19 | 4.86 | 48.19 |
| red 7 (e) | 1.21 | 1.21 | 1.21 | 1.21 | 1.21 | 1.21 |
| Disteardimonium hectorite | 10 | 10 | 10 | 10 | 10 | 10 |
| (and) propylene carbonate (f) | | | | | | |
| (10%/3%) (Bentone Gel, | | | | | | |
| Elementis) | | | | | | |
| (a):(b) | 1:2.33 | 1:1 | 2.33:1 | — | — | — |

¹MQ is trimethylsiloxysilicate (Momentive). PSPA is nylon-611/dimethicone copolymer (Dow Corning).

### Preparation of Liquid Lipsticks

Polymer solutions, wax, and a portion of isododecane were blended under high shear at 100° C. until all materials were completely blended. The solution temperature was brought down to 65° C. and Bentone Gel was added under high shear. Once the mixture became homogenous, pigment solution, and the final amount of isododecane was added to the mixture and blended until homogenous.

### Bioskin Adhesion and Resistance Testing

The compositions were tested for their wear on bioskin (Bio Skin Plate Regular Type 195×130×5 (T) mm #white). Samples were deposited onto the bioskin surface using a 3 mm draw down bar. The samples were allowed to dry for two hours, and then soaked in olive oil for 1 minute. The samples were each individually wiped with a kimwipe 15 times. A rating from 1-5 was given for how well the sample resisted removal. A rating of 1 indicated complete resistance to removal and a rating of 5 indicated complete removal of the sample. The results of this test are provided in Table 4 below.

As is shown in Table 4, the compositions of Examples 1 and 2 had good resistance to removal from the bioskin when soaked and rubbed with olive oil. This result was also demonstrated for the Control 1 and Comparative 2 compositions as well. The compositions of Comparative 1 and Control 2 exhibited more removal.

### Flake Testing

Samples were additionally tested for their cohesive properties as follows. Samples were deposited onto the bioskin surface using a 3 mm draw down bar. The samples were allowed to dry for two hours after which they were stretched. A rating scale from 1-5 was used to assess the degree of sample flaking upon stretching. A rating of 1 indicated no flaking and a rating of 5 indicated complete flaking (removal). The results of this test are also shown in Table 4 below. Comparative 1 and Examples 1 and 2 had good resistance to flaking as well as Control 1 and Comparative 2. Control 2 completely flaked when stretched.

TABLE 4
Gloss Testing

| | | | | | | Com- |
|---|---|---|---|---|---|---|
| | Compar- | Exam- | Exam- | | | parative |
| | ative 1 | ple 1 | ple 2 | Control 1 | Control 2 | 2 |
| Bioskin | 5 | 1 | 2 | 2 | 3 | 1 |
| Wear | | | | | | |
| Testing | | | | | | |
| Flake | 1 | 1 | 1 | 1 | 5 | 2 |
| Testing | | | | | | |

### Gloss Testing

The gloss of the inventive compositions as well as of the Comparators and Controls was measured using a BYK Gardner micro gloss meter following the ASTM Standard Test Method for determining Gloss as described at http://www.astm.org/Standards/D523.htm.

Sample films were prepared using a 3 mm drawdown bar onto contrast paper. The films were allowed to dry over the course of 2-4 hours after which the gloss of the resulting films was measured the gloss meter. The measurements are reported in gloss units (GU) which represent the ratio of reflected to incident light of the films compared to that for a standard. Low gloss is a GU measurement less than 10 at a measurement angle of 60 degrees. Medium gloss is a GU measurement from 10-70 GU at a measurement angle of 60 degrees. High gloss is a GU measurement above 70 at a measurement angle of 60 degrees. The results of this test are reported in Table 5 below. The results in Table 5 are an average of three trials.

TABLE 5
Gloss values of lipsticks

| | Comparative | Example | Example | Control | Control |
|---|---|---|---|---|---|
| | 1 | 1 | 2 | 1 | 2 |
| 20 degree | 11.8 | 11.1 | 41.5 | 8 | 3.8 |
| 60 degree | 46.1 | 45.1 | 72.1 | 38.1 | 25.3 |
| 85 degree | 72 | 78.9 | 85 | 63.1 | 61.8 |

As shown in Table 5 above, Control samples demonstrated medium (25 GU-38 GU) gloss values at measurement angle of 60 degrees. The gloss value at the same measurement angle for inventive compositions of Examples 1 and 2 was medium to high (45 GU-72 GU). Example 2 (ratio of (a):(b)=2.33:1) had the highest gloss value at all angles measured.

Comparison with Currently Marketed MQ-Based Products

The performance of the compositions for the invention was tested against currently marketed long wear lip products. Marketed products designated as being “two step” as well as single step products were evaluated.

For two step products the basecoat was evaluated for the gloss properties. All samples were prepared in the same fashion as described above. The results of this comparison are shown in Table 6 below.

TABLE 6
Comparison with Commercial Products

| Sample | 20° C. | 60° C. | 85° C. |
|---|---|---|---|
| Example 2 | 41.5 | 72.1 | 85 |
| MKT1 ¹ Basecoat (MQ + PSPA) | 5.4 | 21.2 | 32.2 |
| MKT2 Basecoat (MQ + dimethicone) | 0 | 1.7 | 10.5 |
| MKT3 Basecoat (MQ + dimethicone) | 0.3 | 3.2 | 21.3 |
| MKT4² (MQ + dimethicone) | 0.4 | 3.8 | 16.3 |
| MKT5 (MQ + dimethicone) | 0.2 | 1.8 | 8.9 |
| MKT6 (MQ + dimethicone) | 0.1 | 0.7 | 2.8 |

¹ MKT 1-3 are all two-step products. The base coat of these products was considered for gloss.
²MKT 3-6 are single step products.

As is shown in Table 6 above, the composition of applicants' Example 2 had the best gloss values of all of the compositions tested at each of the three measured angles. The compositions of the invention thus afford a long wear lipstick composition that has increased shine as compared with MQ-based lip compositions without the use of additional non-volatile solvents.

What is claimed is:

1. A liquid lipstick composition comprising:
(a) from about 15% to about 40% by weight of at least one silicone acrylate copolymer;
(b) from about 5% to about 25% by weight of at least one polypropylsilsesquioxane film forming resin;
(c) from about 0.1% to about 2.0% by weight of at least one polypropylsilsesquioxane wax substituted with alkyl units having at least 30 carbons;
(d) from about 15% to about 60% by weight of at least one volatile hydrocarbon solvent;
(e) optionally least one colorant; and
(f) optionally at least one viscosity increasing agent;
wherein the weight percent ratio of the silicone acrylate copolymer (a) to the at least one polypropylsilsesquioxane film forming resin (b) is from about 1:1 to about 4:1; the total weight percent of the at least one silicone acrylate copolymer (a) plus the at least one polypropylsilsesquioxane film forming resin (b) is equal to or greater than about 30%; the weights being relative to the total weight of the composition.

2. The composition of claim 1 wherein the sum of the weight percent of the at least one silicone acrylate copolymer (a) and the at least one polypropylsilsesquioxane film forming resin (b) is from about 35% to about 55% by weight relative less to the total weight of the composition.

3. The composition of claim 2 wherein the silicone acrylate copolymer (a) is selected from acrylates/dimethicone copolymer and acrylates/polytrimethylsiloxymethacrylate copolymer, and mixtures thereof.

4. The composition of claim 3 wherein the silicone acrylate copolymer (a) is selected from cyclopentasiloxane (and) acrylates/dimethicone copolymer, butyl acetate (and) acrylates/dimethicone copolymer, methyl trimethicone (and) acrylates/dimethicone copolymer, isododecane (and) acrylate /dimethicone copolymer, cyclopentasiloxane (and) acrylates/polytrimethylsiloxymethacrylate copolymer, isododecane (and) acrylates/polytrimethylsiloxymethacrylate copolymer, and mixtures thereof.

5. The composition of claim 2 wherein the at least one polypropylsilsesquioxane film forming resin (b) has the general formula RnSiO(4−n)/2; wherein R is independently chosen from a hydrogen atom and a monovalent hydrocarbon group comprising 3 carbon atoms, wherein more than 80 mole % of R are propyl groups; n is a value from 1.0 to 1.4; more than 60 mole % of the copolymer comprises RSiO3/2 units; said film forming resin having a hydroxyl or alkoxy content from 0.2 to 10% by weight of the resin, a molecular weight from about 5000 to about 30,000 and a Tg from about −5° C. to about 5° C.

6. The composition of claim 5 wherein the number average molecular weight of the polypropylsilsesquioxane wax substituted with alkyl units having at least 30 carbons (c) is from about 750 to about 10,000.

7. The composition of claim 6 wherein the at least one polypropylsilsesquioxane wax is selected from a C30-45 alkyldimethylsilyl polypropylsilsesquioxane (and) paraffin wax having a Tm from about 60° C. to about 80° C.

8. The composition of claim 7 wherein the volatile hydrocarbon solvent (d) is selected from isododecane, isooctane, isodecane, isohexadecane, and mixtures thereof.

9. The composition of claim 8 wherein the silicone acrylate copolymer (a) has a glass transition temperature (Tg) above 20° C.

10. The composition of claim 9 that includes a colorant.

11. The composition of claim 8 that includes a viscosity increasing agent.

12. The composition of claim 11 wherein the viscosity increasing agent is a gelling agent.

13. The composition of claim 12 wherein the gelling agent is selected from a modified clay.

14. The composition of claim 13 wherein the modified clay is selected from a hectorite modified with distearyldimethylammonium chloride or modified with stearyldimethylbenzoylammonium chloride.

15. The composition of claim 14 wherein the gelling agent is disteardimonium hectorite.

16. The composition of claim 15 wherein the ratio of the silicone acrylate copolymer (a) to the at least one polypropylsilsesquioxane film forming resin (b) is about 2.3:1.

17. A liquid lipstick composition comprising:
(a) from about 15% to about 40% by weight of at least one silicone acrylate copolymer;
(b) from about 5% to about 25% by weight of at least one polypropylsilsesquioxane film forming resin;
(c) from about 0.1% to about 2% by weight of at least one polypropylsilsesquioxane wax substituted with alkyl units having at least 30 carbons;
(d) from about 15% to about 60% by weight of at least one volatile hydrocarbon solvent;
(e) from about 0.5% to about 18% of least one colorant; and
(f) from about 1% to about 20% at least one viscosity increasing agent;
wherein the weight percent ratio of the silicone acrylate copolymer (a) to the at least one polypropylsilsesquioxane film forming resin (b) is from about 1.5:1 to about 4:1; the total weight percent of the at least one silicone acrylate copolymer (a) plus the at least one polypropylsilsesquioxane film forming resin (b) is from about 35% to about 55%%; the weights being relative to the total weight of the composition.

18. The composition of claim 16 wherein the ratio of the silicone acrylate copolymer (a) to the at least one polypropylsilsesquioxane film forming resin (b) is from about 1.1 to about 3:1, by weight.

19. A liquid lipstick composition comprising:
(g) from about 20% to about 29% by weight of at least one silicone acrylate copolymer;
(h) from about 10% to about 22% by weight of at least one polypropylsilsesquioxane film forming resin;
(i) from about 0.4% to about 1% by weight of at least one polypropylsilsesquioxane wax substituted with alkyl units having at least 30 carbons;
(j) from about 4% to about 11% by weight of at least one volatile hydrocarbon solvent;
(k) from about 3% to about 15% of least one colorant; and
(l) about 10% at least one viscosity increasing agent;
wherein the weight percent ratio of the silicone acrylate copolymer (a) to the at least one polypropylsilsesquioxane film forming resin (b) is from about 1:1 to about 2.3:1; the total weight percent of the at least one silicone acrylate copolymer (a) plus the at least one polypropylsilsesquioxane film forming resin (b) is from about 35% to about 55%%; the weights being relative to the total weight of the composition.

20. A method of making up lips comprising applying to the lips a solid lipstick composition comprising:
(a) from about 15% to about 40% by weight of at least one silicone acrylate copolymer;
(b) from about 5% to about 25% by weight of at least one polypropylsilsesquioxane film forming resin;
(c) from about 0.1% to about 4% by weight of at least one polypropylsilsesquioxane wax substituted with alkyl units having at least 30 carbons;
(d) from about 15% to about 60% by weight of at least one volatile hydrocarbon solvent;
(e) optionally least one colorant; and
(f) optionally at least one viscosity increasing agent;
wherein the weight percent ratio of the silicone acrylate copolymer (a) to the at least one polypropylsilsesquioxane film forming resin (b) is from about 1:1 to about 4:1; the total weight percent of the at least one silicone acrylate copolymer (a) plus the at least one polypropylsilsesquioxane film forming resin (b) is equal to or greater than about 30%; the weights being relative to the total weight of the composition.
